(19) United States
(12) Patent Application Publication
(10) Pub. No.: US 2009/0304720 A1
(43) Pub. Date: Dec. 10, 2009

(54) Active Agent-Loaded Nanoparticles Based On Hydrophilic Proteins

(75) Inventors: Jörg Kreuter, Bad Homburg v.d.H (DE); Klaus Langer, Schöneck (DE); Kerstin Michaelis, Frankfurt (DE); Telli Hekmatara, Frankfurt (DE); Sebastian Dreis, Frankfurt (DE)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG, Andernach (DE)

(21) Appl. No.: 12/225,151

(22) PCT Filed: Feb. 27, 2007
(86) PCT No.: PCT/EP2007/001675

§ 371 Date: Jun. 9, 2009

(30) Foreign Application Priority Data
Mar. 14, 2006 (DE) 10 2006 011 507.4

(51) Int. Cl. A61K 47/48; A61K 39/395; A61K 38/43; A61K 38/22; A61K 31/704; A61K 31/4375; A61K 31/451

(52) U.S. Cl. 424/179.1; 424/94.1; 514/2; 514/12; 514/34; 514/279; 514/327; 977/773; 977/906

(57) ABSTRACT

Active agent-loaded nanoparticles that are based on a hydrophilic protein or a combination of hydrophilic proteins, and methods for producing the nanoparticles and the use thereof. Functional proteins or peptide fragments are bound to the nanoparticles via polyethylene glycol-α-maleimide-ω-NHS esters.

### CROSS-REFERENCE TO RELATED APPLICATIONS

This application is a National Stage application of International Application No. PCT/EP2007/001675, filed on Feb. 27, 2007, which claims priority of German application number 10 2006 011 507.4, filed on Mar. 14, 2006, both of which are incorporated herein by reference in their entireties.

### BACKGROUND OF THE INVENTION

1. Field of the Invention

The present invention relates to active agent-loaded nanoparticles that are based on a hydrophilic protein or a combination of hydrophilic proteins and in which functional proteins or peptide fragments are bound to the nanoparticles via polyethylene glycol-α-maleimide-ω-NHS esters. More particularly, the invention relates to active agent-loaded nanoparticles that are based on at least one hydrophilic protein and in which functional proteins or peptide fragments, preferably an apolipoprotein, are bound to the nanoparticles via polyethylene glycol-α-maleimide-ω-NHS esters, in order to transport the pharmaceutically or biologically active agent across the blood-brain barrier.

2. Description of the Prior Art

The term “nanoparticles” is understood to mean particles having a size of between 10 nm and 1000 nm and made up of artificial or natural macromolecular substances to which drugs or other biologically active materials may be bound by covalent, ionic or adsorptive linkage, or into which these substances may be incorporated.

By means of certain nanoparticles, it is possible to transport hydrophilic drugs, which by themselves are not able to cross the blood-brain barrier, across said barrier so that these hydrophilic drugs can become therapeutically active in the central nervous system (CNS).

For example, it has been possible to transport a number of drugs across the blood-brain barrier by means of polybutylcyanoacrylate nanoparticles which are coated with polysorbate 80 (TWEEN® 80) or other tensides, and which produce a significant pharmacological effect through their action in the central nervous system. Examples of drugs that are administered with such polybutylcyanoacrylate nanoparticles include dalargin, an endorphin hexapeptide, loperamide and tubocuarine, the two NMDA receptor antagonists MRZ 2/576 and MRZ 2/596, respectively, of the company Merz, Frankfurt, as well as the antineoplastic active agent doxorubicin.

The mechanism of transport of these nanoparticles across the blood-brain barrier is possibly based on apolipoprotein E (ApoE) being adsorbed by the nanoparticles via the polysorbate 80 coating. Presumably, these particles thereby mimic lipoprotein particles, which are recognized and bound by receptors of the brain endothelial cells, which ensure the supply of lipids to the brain.

The polybutylcyanoacrylate nanoparticles known to cross the blood-brain barrier, however, have drawbacks in that polysorbate 80 is not of physiological origin and in that the transport of the nanoparticles across the blood-brain barrier may possibly be due to a toxic effect of polysorbate 80. In addition, the known polybutylcyanoacrylate nanoparticles also have the disadvantage that the binding of the ApoE takes place only by adsorption. Thereby, the nanoparticle-bound ApoE is present in equilibrium with free APoE, and, after injection into the body, rapid desorption of the ApoE from the particles may occur. Furthermore, many drugs do not bind to polybutylcyanoacrylate nanoparticles to a sufficient extent and can therefore not be transported across the blood-brain barrier with this carrier system.

To overcome these disadvantages, WO 02/089776 A1 proposes nanoparticles of human serum albumin (HSA nanoparticles), to which biotinylated apolipoprotein E is bound via an avidin-biotin system or an avidin derivative. Following intravenous injection, these HSA nanoparticles can transport drugs that are adsorptively or covalently bound, as well as drugs that are incorporated in the particle matrix, across the blood-brain barrier (BBB). In this manner, active agents which otherwise are not able to cross that barrier for biochemical, chemical or physicochemical reasons, can be utilised for pharmacological and therapeutic applications in the CNS.

The avidin-biotin system does have various drawbacks, however. For example, its use is complex as regards the production of the nanoparticles and can, in addition, lead to immunological or other side effects. Furthermore, particle systems that comprise an avidin-biotin system tend to agglomerate when stored for prolonged periods, which leads to an increase in mean particle size and has an adverse effect on the efficiency of the particles.

### SUMMARY OF THE PRESENT INVENTION

The task underlying the present invention thus was to provide nanoparticles by means of which drugs which, for biochemical, chemical or physicochemical reasons, are not able to cross the blood-brain barrier can be supplied to the CNS, without these nanoparticles having the disadvantages of the polybutylcyanoacrylate nanoparticles known from the prior art and of the HSA nanoparticles comprising an avidin-biotin system.

This task is solved by nanoparticles that are based on a hydrophilic protein or a combination of hydrophilic proteins, comprise at least one pharmacologically acceptable and/or biologically active agent, and to which an apolipoprotein serving as a functional protein is bound via polyethylene glycol-α-maleimides-ω-NHS esters.

The hydrophilic protein, or at least one of the hydrophilic proteins, on which the nanoparticles according to the invention are based, preferably belongs to the group of proteins which comprises serum albumins, gelatine A, gelatine B and casein. Hydrophilic proteins of human origin are more preferred. Most preferably, the nanoparticles are based on human serum albumin.

The bifunctional polyethylene glycol-α-maleimide-ω-NHS esters comprise a maleimide group and an N-hydroxysuccinimide ester, between which there is a polyethylene glycol chain of defined length. Preferably, the functional protein or peptide fragment is coupled to the hydrophile protein via polyethylene glycol-α-maleimide-ω-NHS esters which comprise a polyethylene glycol chain having a mean molecular weight of 3400 Da or 5000 Da.

The apolipoprotein bound to the hydrophilic protein via the polyethylene glycol-α-maleimide-ω-NHS ester is preferably selected from the group consisting of apolipoprotein E, apolipoprotein B (ApoB) and apolipoprotein A1 (ApoA1).

In other preferred embodiments of the nanoparticles according to the invention, the functional protein is not an apolipoprotein but is selected from the group of proteins which consists of antibodies, enzymes and peptide hormones. However, it is also possible to couple almost any desired peptide fragment, preferably a peptide fragment from the group of the functionally active fragments of the afore-mentioned functional proteins, to the nanoparticles via polyethylene glycol-α-maleimide-ω-NHS esters.

The subject matter of the present invention therefore are active agent-loaded nanoparticles which are based on a hydrophilic protein or a combination of hydrophilic proteins and wherein the nanoparticles comprise at least one functional protein or peptide fragment which is bound to the hydrophilic protein or the hydrophilic proteins, via polyethylene glycol-α-maleimide-ω-NHS esters.

Loading of the nanoparticles with the active agent to be transported may be accomplished by adsorption of the active agent to the nanoparticles, incorporation of the active agent into the nanoparticles, or by covalent or complexing linkage via reactive groups.

In principle, the nanoparticles according to the invention may be loaded with almost any desired active agent/drug. Preferably, however, the nanoparticles are loaded with active agents which themselves are not able to cross the blood-brain barrier. More preferably, the active agents belong to the groups of the cytostatic agents, antibiotics, antiviral substances, and drugs which are active against neurologic diseases, for example from the group comprising analgesic agents, nootropics, anti-epileptics, sedatives, psychotropic drugs, pituitary hormones, hypothalamic hormones, other regulatory peptides and inhibitors thereof, this list by no means being definitive. Most preferably, the active agent is selected from the group which comprises dalargin, loperamide, tubocuarine and doxorubicin.

The nanoparticles according to the invention are advantageous in that it is not necessary to utilise the avidin-biotin system, which possibly causes side effects, to couple the functional proteins or the peptide fragments thereof to the hydrophilic protein of the particles.

Preferably, the nanoparticles according to the invention are produced by initially converting an aqueous solution of the hydrophilic protein or of the hydrophilic proteins to nanoparticles by a desolvation process, and by subsequently stabilising said nanoparticles by crosslinking.

Desolvation from the aqueous solvent is preferably accomplished by addition of ethanol. In principle, it is also possible to achieve desolvation by adding other water-miscible non-solvents for hydrophilic proteins, such as acetone, isopropanol or methanol. Thus, gelatine was successfully desolvatised as a starting protein by addition of acetone. Desolvation of proteins dissolved in aqueous phase is likewise possible by adding structure-forming salts such as magnesium sulfate or ammonium sulfate. This is called salting out.

Suitable crosslinking agents for stabilising the nanoparticles are bifunctional aldehydes, preferably glutaraldehyde, as well as formaldehyde. Furthermore, it is possible to crosslink the nanoparticle matrix by thermal processes. Stable nanoparticle systems were obtained at 60° C. for periods of more than 25 hours, or at 70° C. for periods of more than 2 hours.

The functional groups located on the surface of the stabilised nanoparticles (amino groups, carboxyl groups, hydroxyl groups) can be used for direct covalent conjugation of apolipoproteins. These functional groups can be bound via heterobifunctional “spacers”, being reactive to both amino groups and free thiol groups, to an apolipoprotein in which free thiol groups have previously been introduced.

To produce the nanoparticles according to the invention, the amino groups of the particle surface are converted with the heterobifunctional polyethylene glycol (PEG)-based crosslinker polyethylene glycol-α-maleimide-ω-NHS ester. In this process, the succinimidyl groups of the polyethylene glycol-α-maleimide-ω-NHS ester react with the amino groups of the particle surface, releasing N-hydroxysuccinimide. By means of this reaction it is possible to introduce PEG groups on the particle surface which, in turn, comprise maleimide groups at the other end of the chain which can react with a thiolated substance, thereby forming a thioether.

The polyethylene glycol chain of the polyethylene glycol-α-maleimide-ω-NHS ester preferred for producing the nanoparticles according to the invention has a mean molecular weight of 3400 Da (NHS-PEG3400-Mal). However, in principle, it is also possible to utilise polyethylene glycol-α-maleimide-ω-NHS esters that comprise shorter or longer polyethylene glycol chains, for example a polyethylene glycol chain having a mean molecular weight of 5000 Dalton.

For producing the nanoparticles according to the invention, the apolipoprotein, the functional protein or the peptide fragment which is to be coupled are thiolated by conversion with 2-iminothiolane. The free amino groups of the proteins or peptide fragments are used for this conversion.

After each reaction step, the particle systems are purified by repeatedly centrifuging and redispersing in aqueous solution. Following the conversion, the respective dissolved protein is, in principle, separated from the low-molecular reaction products by size exclusion chromatography.

The preferred method for producing the active agent-loaded nanoparticles which are based on a hydrophilic protein or on a combination of hydrophilic proteins and are modified with functional proteins or peptide fragments comprises the following steps:
desolvating an aqueous solution of a hydrophile protein or a combination of hydrophile proteins,
stabilising the nanoparticles produced by the desolvation by crosslinking,
converting the amino groups on the surface of the stabilised nanoparticles with polyethylene glycol-α-maleimide-ω-NHS ester,
thiolating the functional proteins or peptide fragments; and
covalently attaching the thiolated proteins or peptide fragments to the nanoparticles converted with polyethylene glycol-α-maleimide-ω-NHS ester.

To mediate pharmacological effects, pharmaceutically or biologically active substances (active agents) can be incorporated in the particles. In that case, binding of the active agent may be accomplished by covalent, complexing, as well as by adsorptive linkage.

Following covalent binding of the thiolated apolipoprotein or of another thiolated functional protein or peptide fragment, the PEG-modified nanoparticles are preferably adsorptively loaded with the active agent.

In a particularly preferred method the hydrophilic protein, or at least one of the hydrophilic proteins, is selected from the group of proteins comprising serum albumins, gelatine A, gelatine B and casein and comparable proteins, or a combination of these proteins. Most preferably, hydrophile proteins of human origin are used for the production.

The inventive nanoparticles of a hydrophile protein or a combination of hydrophile proteins having apolipoprotein E bound thereto are suitable for transporting pharmaceutically or biologically active agents that otherwise would not cross the blood-brain barrier, in particular hydrophile active agents, across the blood-brain barrier and to induce pharmacological effects. Preferred active agents belong to the groups of the cytostatic agents, antibiotics, and drugs which are active against neurologic diseases, for example the group comprising analgesic agents, nootropics, anti-epileptics, sedatives, psychotropic drugs, pituitary hormones, hypothalamic hormones, other regulatory peptides and inhibitors thereof. Examples of such active agents are dalargin, loperamide, tubocuarine, doxorubicin, or the like.

### BRIEF DESCRIPTION OF THE DRAWINGS

FIG. 1: Graphic representation of the analgesic effect (maximal possible effect, MPE) following intravenous application of loperamide-loaded HSA nanoparticles modified with apolipoprotein via polyethylene glycol-α-maleimide-ω-NHS esters.

### DETAILED DESCRIPTION OF THE PRESENT INVENTION

Hence, the nanoparticles described herein, which have been loaded with active agent and modified with apolipoprotein, are suitable for treating a large number of cerebral diseases. To this end, the active agents bound to the carrier system are selected in accordance with the respective therapeutic aim. The carrier system suggests itself above all for those active substances which show no passage or an insufficient passage across the blood-brain barrier. Substances which are considered suitable as active agents are cytostatic agents for the therapy of cerebral tumours, active agents for the therapy of viral infections in the cerebral region, e.g. HIV infections, but also active agents for the therapy of dementia affections, to mention but a few application areas.

Hence, another subject matter of the invention is the use of the nanoparticles according to the invention for producing medicaments; more particularly the use of nanoparticles according to the invention in which the functional protein is an apolipoprotein for producing a medicament for the treatment of cerebral diseases and, respectively, the use of such proteins for treating cerebral diseases, as these nanoparticles can be utilised for transporting pharmaceutically or biologically active agents across the blood-brain barrier.

### Example

To produce HSA nanoparticles by desolvation, 200 mg of human serum albumin was dissolved in 2.0 ml of a 10 mM NaCl solution, and the pH of this solution was adjusted to a value of 8.0. Under stirring, 8.0 ml of ethanol were added to this solution by drop-wise addition, at a rate of 1.0 ml/min. This desolvation step lead to the formation of HSA nanoparticles having a mean particle size of 200 nm.

The nanoparticles were stabilised by adding 235 μl of an 8% glutaraldehyde solution. Following an incubation period of 12 h, the nanoparticles were purified by centrifuging and redispersing three times, initially in purified water and subsequently in PBS buffer (pH 8.0).

To activate the nanoparticles, 500 μl of a solution of the crosslinker NHS-PEG3400-Mal (60 mg/ml in PBS buffer 8.0) were added to 2.0 ml of the nanoparticle suspension (20 mg/ml in PBS buffer) and incubated at room temperature for 1 hour, under agitation. After the incubation period, the PEG-modified nanoparticles were purified with purified water, as described above. These steps yielded PEGylated HSA nanoparticles which, via maleimide groups of the PEG derivative applied to the surface, had reactivity for free thiol groups.

For covalent binding of an apolipoprotein, initially, free thiol groups were introduced in the structure thereof. To this end, 500 μg of the apolipoprotein were dissolved in 1.0 ml of TEA buffer (pH 8.0), and 2-iminothiolane (Traut's reagent) was added in a 50-fold molar excess. Following a reaction period of 12 hours at room temperature, the thiolated apolipoprotein was purified by size exclusion chromatography via a dextran desalting column (D-SALT® Column), and low-molecular reaction products were separated in the process.

For covalent conjugation of the thiolated apolipoprotein to HSA nanoparticles, 500 μg of the thiolated apolipoprotein were added to 25 mg of the PEG-modified HSA nanoparticles, and this mixture was incubated at room temperature for 12 hours. After that reaction period, non-reacted apolipoprotein was removed by centrifuging and redispersing the nanoparticles. In the final purification step, the apolipoprotein-modified HSA nanoparticles were taken up in ethanol 2.6% by volume.

In separate samples, apolipoprotein E, apolipoprotein B and apolipoprotein A1 were thiolated and coupled to HSA nanoparticles.

For loading the nanoparticles with the model drug loperamide, 6.6 mg loperamide in ethanol 2.6% by volume were added to 20 mg of the ApoE-modified nanoparticles and incubated for 2 hours. After that time, non-bound drug was separated by centrifuging and redispersing; the resultant loperamide-loaded apolipoprotein-modified HSA nanoparticles were taken up in water for injection purposes, and the particle content was adjusted by diluting with water to 10 mg/ml. The nanoparticles were used in animal experiments, to examine their suitability for the transport of active agents across the blood-brain barrier.

Loperamide as opioid, which in dissolved form is not able to cross the blood-brain barrier (BBB), is a particularly suitable model drug for a corresponding carrier system for crossing the BBB. An analgesic effect occurring after application of a loperamide-containing preparation provides direct proof that the substance has accumulated in the central nervous system and hence that the BBB has been overcome.

A typical nanoparticulate preparation used in the animal experiment contained 10.0 mg/ml nanoparticles, 0.7 mg/ml loperamide and 190 μg/ml ApoE.

The compositions of the ready-to-apply nanoparticulate preparations (total volume 2.0 ml) for the animal experiments were as follows:

| | | |
|---|---|---|
| 1. | 10.0 mg/ml | apolipoprotein-modified HSA nanoparticles |
| 2. | 190.0 μg/ml | apolipoprotein, covalently bound |
| 3. | 0.7 mg/ml | loperamide (adsorptively bound to the nanoparticles) |

| | |
|---|---|
| 4. | water for injection purposes.. |

The preparations were applied intravenously to mice, at a dosage of 7.0 mg/kg loperamide. Based on an average body weight of a mouse of 20 g, the animals received an application amount of 200 μl of the above-mentioned preparation.

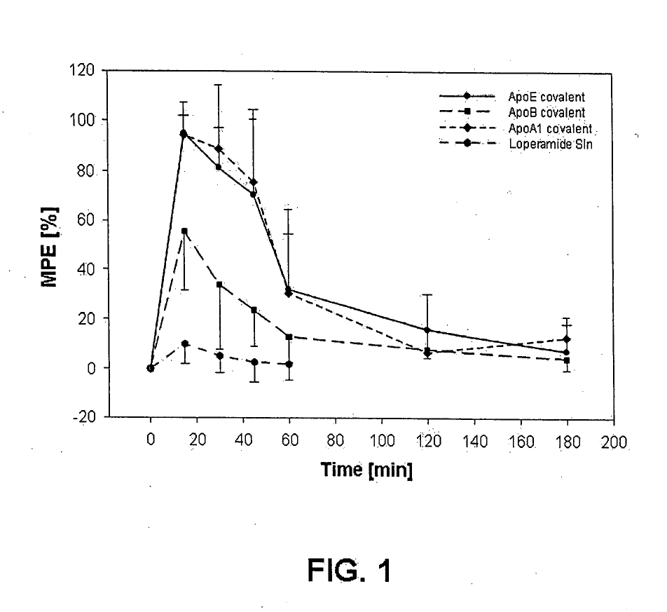

With the aid of this system, the analgesic effects shown in FIG. 1 were achieved after intravenous injection using the above-mentioned active agent loperamide. Analgesia (Nociceptive Response) was detected by the tail-flick test, wherein a hot beam of light is projected onto the tail of the mouse and the time that passes until the mouse flicks away its tail is measured. After ten seconds (=100% MPE) the experiment is discontinued so as not to cause injury to the mouse. Negative MPE values occur in those cases where following administration of the preparation, the mouse flicks away its tail earlier than before the treatment.

As a comparison, a loperamide solution 0.7 mg/ml in 2.6%-vol. ethanol was used. The free substance loperamide itself exhibits no analgesic effect, due to lack of transport across the blood brain barrier.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. Active agent-loaded nanoparticles based on a hydrophilic protein or a combination of hydrophilic proteins, wherein said nanoparticles comprise at least one functional protein or peptide fragment which is bound to the hydrophile protein or the hydrophile proteins via polyethylene glycol-α-maleimide-ω-NHS esters.

2. The nanoparticles according to claim 1, wherein the hydrophile protein or at least one of the hydrophile proteins is selected from the group consisting of serum albumins, gelatine A, gelatine B and casein.

3. The nanoparticles according to claim 1, wherein the hydrophilic protein or at least one of the hydrophilic proteins is of human origin.

4. The nanoparticles according to claim 1, wherein the functional protein or peptide fragment is selected from the group consisting of apolipoproteins, antibodies, enzymes, hormones, cytostatic agents, antibiotics, and fragments thereof.

5. The nanoparticles according to claim 4, wherein the functional protein is selected from the group consisting of apolipoprotein A1, apolipoprotein B and apolipoprotein E.

6. The nanoparticles according to claim 1, wherein the polyethylene glycol-α-maleimide-ω-NHS ester is selected from the group consisting of the polyethylene glycol-α-maleimide-ω-NHS esters that comprise a polyethylene glycol chain having a mean molecular weight of 3400 Da or 5000 Da.

7. The nanoparticles according to claim 1, wherein the nanoparticles are loaded with active agent by a process selected from the group consisting of adsorption, incorporation, covalent linkage via reactive groups and complexing linkage via reactive groups.

8. The nanoparticles according to claim 1, wherein the active agent is selected from the group consisting of cytostatic agents, antibiotics, antiviral substances, analgesic agents, nootropics, anti-epileptics, sedatives, psychotropic drugs, pituitary hormones, hypothalamic hormones, other regulatory peptides and inhibitors thereof.

9. The nanoparticles according to claim 8, wherein the active agent is selected from the group consisting of dalargin, loperamide, tubocuarine and doxorubicin.

10. A method for producing active agent-loaded nanoparticles which are based on a hydrophilic protein or on a combination of hydrophilic proteins and are modified with functional proteins or peptide fragments, said method comprising the following steps:
desolvating an aqueous solution of a hydrophile protein or a combination of hydrophile proteins to produce nanoparticles, said nanoparticles comprising amino groups on the surface of the nanoparticles;
crosslinking the nanoparticles produced by the desolvation step to stabilize the nanoparticles;
converting the amino groups on the surface of the stabilised nanoparticles with polyethylene glycol-α-maleimide-ω-NHS ester;
thiolating the functional proteins or peptide fragments; and
covalently attaching or binding the thiolated proteins or peptide fragments to the nanoparticles converted with polyethylene glycol-α-maleimide-ω-NHS ester.

11. The method according to claim 10, further comprising the step of adsorptively loading the nanoparticles with active agent, following the step of binding the thiolated protein or peptide fragment.

12. The method according to claim 10, wherein the hydrophilic protein is at least one protein selected from the group consisting of serum albumins, gelatine A, gelatine B, casein and comparable proteins.

13. The method according to claim 10, wherein the hydrophilic protein is of human origin.

14. The method according to claim 10, wherein the desolvation step comprises stirring and adding a water-miscible non-solvent for hydrophilic proteins, or salting-out.

15. The method according to claim 14, wherein the water-miscible non-solvent for hydrophilic proteins is selected from the group comprising ethanol, methanol, isopropanol and acetone.

16. The method according to claim 10, wherein the step of stabilizing the nanoparticles comprises using thermal processes or bifunctional aldehydes or formaldehyde.

17. The method according to claim 16, wherein said bifunctional aldehyde is glutaraldehyde.

18. The method according to claim 10, wherein the polyethylene glycol-α-maleimide-ω-NHS ester is selected from the group consisting of the polyethylene glycol-α-maleimide-ω-NHS esters that comprise a polyethylene glycol chain having a mean molecular weight of 3400 Da or 5000 Da.

19. The method according to claim 10, wherein the agent which modifies thiol groups is 2-iminothiolane.

20. The method according to claim 10, wherein the active agents are selected from the group consisting of cytostatic agents, antibiotics, antiviral substances, analgesic agents, nootropics, anti-epileptics, sedatives, psychotropic drugs, pituitary hormones, hypothalamic hormones, other regulatory peptides and inhibitors thereof.

21. The method according to claim 20, wherein the active agents are selected from the group consisting of dalargin, loperamide, tubocuarine and doxorubicin.

22. Use of active agent-loaded nanoparticles comprising apolipoprotein that is bound to hydrophilic proteins via polyethylene glycol-α-maleimide-ω-NHS esters, for transporting pharmaceutically or biologically active agents across the blood-brain barrier.

23. The use according to claim 22, wherein the hydrophilic protein is at least one protein selected from the group consisting of serum albumins, gelatine A, gelatine B, casein and comparable proteins.

24. The use of active agent-loaded nanoparticles according to claim 22, wherein at least one of the hydrophilic proteins is of human origin.

25. The use of active agent-loaded nanoparticles according to claim 22, wherein the active agents are selected from the group consisting of cytostatic agents, antibiotics, antiviral substances, analgesic agents, nootropics, anti-epileptics, sedatives, psychotropic drugs, pituitary hormones, hypothalamic hormones, other regulatory peptides and inhibitors thereof.

26. The use of active agent-loaded nanoparticles according to claim 25, wherein the active agents are selected from the group consisting of dalargin, loperamide, tubocuarine and doxorubicin.

27. The use of active agent-loaded nanoparticles according to claim 22, wherein the nanoparticles are used for treating cerebral affections.

28. The use of active agent loaded nanoparticles according to claim 1, for producing a medicament.

29. The use of active agent loaded nanoparticles according to claim 1, wherein the functional protein is an apolipoprotein, for producing a medicament for treating cerebral affections.

30. The use of active agent loaded nanoparticles according to claim 1, wherein the functional protein is an apolipoprotein, for treating cerebral affections.
